(19) United States
(12) Patent Application Publication
(10) Pub. No.: US 2006/0259001 A1
(43) Pub. Date: Nov. 16, 2006

(54) Disposable nappy

(75) Inventors: Wolfgang Roehrl, Herbrechtingen (DE); Hans-Peter Stupperich, Heidenheim (DE)

Correspondence Address: DREISS, FUHLENDORF, STEIMLE & BECKER, POSTFACH 10 37 62, D-70032 STUTTGART, DE

(73) Assignee: PAUL HARTMANN AG, Heidenheim (DE)

(21) Appl. No.: 10/568,462

(22) PCT Filed: Jul. 23, 2004
(86) PCT No.: PCT/EP04/08219

§ 371 Date: Feb. 15, 2006

(30) Foreign Application Priority Data
Aug. 22, 2003 (DE) 103 38 607.6

(51) Int. Cl. A61F 13/15

(52) U.S. Cl. 604/385.11; 604/395; 604/392

(57) ABSTRACT

The invention relates to a disposable diaper (2) comprising a hip belt (6) creating a hip opening that is closed in the peripheral direction in addition to a main part of the diaper consisting of a front region (14), a rear region (10) and an interlying intermediate region (12). The main part (8) of the diaper can be detachably attached to the hip belt (6) at longitudinal ends of the front region (14) or the rear region (10) thereof, by means of a closing means (18), in such a way that, when the hip belt (6) is applied, a user reaches for the main part of the diaper (8) between the legs, and the free longitudinal end of the main part of the diaper (8) can be detachably fixed to the hip belt (6). The inventive diaper is embodied in such a way that, starting from a longitudinal end region (16) of the main part (8) of the diaper, two expected fracture lines (20) or separating cuts are provided in the main part (8) of the diaper, extending in the direction of the intermediate region (12), such that a central section (22) of the main part (18), arranged between the expected fracture lines (20) or separating cuts can be flapped or folded away from the hip belt (6) along said expected fracture lines 820) or separating cuts, and can be detachably reconnected to the hip belt (6) and/or adjacent regions (26) of the main part of the diaper (8), by means of a second closing means (24), in order to reclose the diaper.

The invention concerns a disposable diaper, in particular for incontinent care, with a hip belt extending in the peripheral direction to form a closed hip opening for the diaper and having at least one location in which it can be opened and closed, and with a main diaper portion having a front region, a rear region, an intermediate crotch region, a back sheet which is liquid impermeable and which faces away from the body, a top sheet which is liquid permeable and faces towards the body, and an absorption body for body fluids, wherein the main diaper portion can be attached, at a longitudinal end of its front region or of its rear region, in a detachable fashion to the hip belt by means of first closing means in such a fashion that a user, with the hip belt applied, can raise the main diaper portion between the legs to attach the free longitudinal end of the main diaper portion to the hip belt in a detachable fashion.

A plurality of disposable diapers of this kind are known in the art. They have the advantage that the user, when applying the diaper, initially fastens the hip belt about the hips and typically closes it in the stomach region. During this procedure, the main diaper portion of the disposable diaper hangs loosely in the downward direction from the rear region which is normally attached to the closed belt. The user then grasps the freely hanging end of the main diaper portion and guides this main diaper portion between the legs in a forward direction to attach the free longitudinal end of the main diaper portion to the hip belt in an inward or outward position and in a detachable fashion. First closing means are provided for this purpose. Clearly, application of the disposable diaper can also be carried out in such a fashion that, after positioning and closing of the hip belt, the free end of the main diaper portion hangs from the front and can be brought from the front towards the rear between the legs of the user to be attached at the rear region to the hip belt in a detachable fashion. Detachable diapers are also known in the art with which the main diaper portion can be completely detached from the hip belt so that, in particular for the case of patients in need of intense nursing care as well as for immobile users, greater flexibility is guarantied for handling of the disposable diaper.

Departing from this prior art, it is the purpose of the present invention to improve the range of applicability of a disposable diaper of this type.

This purpose is achieved in accordance with the invention with a disposable diaper of the above mentioned kind in that two intended breaking lines or separation cuts are disposed in the main diaper portion to extend from a longitudinal end region of the main diaper portion towards the crotch region in such a fashion that a middle section of the main diaper portion, disposed between the intended breaking lines or the separation cuts, can be folded or flapped away from the hip belt along these intended breaking lines or separation cuts in the main diaper portion and can be attached, by means of second closing means, to the hip belt and/or to bordering regions of the main diaper portions in a detachable fashion to once more close the diaper.

The intended breaking lines or separation cuts can extend from the edge of the longitudinal end region or can be separated from this edge. In the latter case, an approximately transversely extending intended breaking line or separation cut can be used in order to permit separation and flapping open of the middle section.

In accordance with the invention, a disposable diaper, in particular for the care of incontinent adults and for use by incontinent adults, is configured in such a fashion that the user or nursing personnel can open the disposable diaper in the above mentioned fashion for purposes of inspection. Towards this end, the first closing means, or at least that portion of the first closing means which fastens the respective region bordering the intended breaking lines or separation cuts of the main diaper portion to the hip belt, remains closed. Only the second closing means, disposed between the main diaper portion and the hip belt, are opened or are initially disposed in an opened state when the disposable diaper is initially applied to the user, i.e. in the packaged state preferentially prior to use, however, in a position which is preferably appropriate for use. The middle section of the main diaper portion, which is bordered or defined by the intended breaking lines or separation cuts, can then be folded or flapped away from the hip belt to facilitate inspection of the inside portion of the diaper. In this manner, one can verify whether or not the diaper is soiled and in need of replacement or can be further used for the acceptance of liquids or solid body discharges. Towards this end, it has turned out to be particularly advantageous when the diaper must only be partially opened in order to release only some of the closing means, while otherwise remaining in a position of use on the body. By means of the supporting function served by these closing means in the closed state, the disposable diaper can be further held in the chosen optimized position on the body of the user which is selected during initial application. Inspection of the diaper is therefore very rapid and does not disturb the user.

The disposable diaper in accordance with the invention also offers a further advantage in that it must not only be used as a literal incontinence article rather, if appropriate, and in a preferred embodiment for use by male users, it is treated as an undergarment with which the disposable diaper can be partially opened in the front region but otherwise remains in a closed state to allow the user to urinate. In this event, the user folds or flaps open the middle section of the main diaper portion at least to a sufficient extent away from the hip belt, that, in the otherwise applied state of the diaper and attached closing means, can urinate in the usual fashion without wetting the diaper. This has turned out to be particularly advantageous for use of the disposable diaper in accordance with the invention by users having only partial impairment of urination control, wherein the user normally senses the urge to urinate and can seek appropriate facilities to the extent possible. In this event, with the hip belt applied, the user can loosen the second closing means so that the front region of the main diaper portion can be folded or flapped away from the hip belt in the downward direction and, in particular for male users, can proceed to urinate in an outward direction in the usual fashion and without wetting the disposable diaper.

The possibility of such use for a disposable diaper has tremendously positive psychological effects on the user which cannot be overemphasized. In this manner, the user looses the sense of having to use a typical incontinence article, even if this is partially or substantially the case. In particular, contact between the physician or the nursing care personnel and the patient is simplified with the configuration of the disposable diaper in accordance with the invention and communication is facilitated as is acceptance for application or advised use of the incontinence article of the kind mentioned here.

In accordance with the invention, a section of the main diaper portion is thereby defined by at least two intended breaking lines or separation cuts. This section has been designated above as the middle section, however must not be precisely in the middle or symmetric with respect to the middle. This middle section, departing from the applied state of the disposable diaper, can be folded or flapped open. The middle section is otherwise advantageously integrally connected to and remains on the main diaper portion, with the exception of the intended breaking lines or separating cuts.

The middle section is therefore intended to provide an inspection opening for the disposable diaper which can once more be closed.

It has turned out to be advantageous when the region of the middle section of the main diaper portion located between the intended breaking lines or the separation cuts has a width in a transverse direction of 5 to 50 cm, in particular 5 to 40 cm, in particular 5 to 30 cm, in particular 5 to 25 cm, in particular 8 to 25 cm, in particular from 10 to 25 cm and in particular from 15 to 25 cm.

In a further configuration of the invention, the width of the middle section varies in the longitudinal section of the disposable diaper and, departing from the longitudinal end region of the main diaper portion, decreases in width towards the crotch region. The anatomical properties are thereby taken into account.

In a further configuration of the invention, the middle section of the main diaper portion is wide in the transverse direction and the regions of the main diaper portion which neighbor the intended breaking locations or the separation cuts and which remain on the body after the middle section is folded away or flapped away, are narrow. The latter neighboring portions of the main diaper portion preferentially have a width in the transverse direction of 1 to 10 cm, in particular 1 to 8 cm, in particular 1 to 7 cm, in particular 1 to 6 cm, in particular 1 to 5 cm, in particular 2 to 5 cm, in particular 3 to 5 cm.

To the extent that the folded away or flapped away middle section of the main diaper portion is solely to be used for purposes of inspection, it has turned out to be sufficient when the intended breaking lines or separation cuts and therefore the longitudinal extension of the folded away or flapped away middle section has a length of approximately 10 cm, in particular 10 to 25 cm. However, when the folded or flapped away middle section is also intended to be used in the above mentioned fashion to facilitate urination with the disposable diaper otherwise remaining in the applied state, it has turned out to be advantageous when the intended breaking lines or separation cuts have a length in the longitudinal direction of 10 to 60 cm, in particular 10 to 50 cm, in particular 10 to 40 cm, in particular 15 to 40 cm, in particular 20 to 40 cm.

In order to maintain the stability of the disposable diaper and in particular the supporting function of the main diaper portion which remains on the body of the diaper in the folded away or flapped away state, it has turned out to be advantageous when the intended breaking lines or separation cuts have a separation in the longitudinal direction of at least 1 cm, in particular of at least 2 cm, in particular of least 3 cm, in particular at least 4 cm, in particular at least 5 cm up to preferentially 20 cm, in particular up to 10 cm from a middle transverse line of the main diaper section.

In a further substantially advantageous improvement of the invention, the second closing means are disposed in such a fashion that they overlap the intended breaking lines or the separation cuts in the main diaper portion. Towards this end, the second closing means can, in particular, be fashioned in the form of tab or a tape-shaped sections which extend over the intended breaking line or separation cut. In a further variation of this concept in accordance with the invention, the second closing means bridge the intended breaking lines or separation cuts along their entire length, thereby visually covering the intended breaking line or separation cuts. This has turned out to be advantageous in the event that the diaper is intended to achieve a certain visual effect and not give the impression of seams or disturbing interruptions. This variation in accordance with the invention has, however, turned out to be particularly advantageous from a purely technical point of view. In this case, the second closing means can be used to reinforce the main diaper portion in the vicinity of the intended breaking line. This means that the intended breaking lines should be configured in such a fashion that they are easily separated by the user without having the danger that the intended breaking lines unintentionally become prematurely separated in view of the supportive and stabilizing effect of the second closing means which bridge over the intended breaking line. In a corresponding fashion, the configuration of the second closing means can also be used to stabilize a separation cut introduced in the main diaper portion. The second closing means bridging the separation cut can therefore be used to maintain the main diaper portion on the associated neighboring locations of the main diaper portion despite location of the separation cut.

Clearly these functions of connecting the middle diaper section to the bordering regions of the main diaper portion during dispensing of the disposable diaper to the user in particular in the packaged state prior to use, can be effected using arbitrary means, in particular gluing means or mechanically binding band sections, tabs or strips. They can, in particular, be completely removed and separated from the disposable diaper for first folding or flapping away of the middle diaper section from the main diaper section. It would also be possible to initially completely remove the stabilizing sections from the main diaper portion in the previously described manner and then preferably rotate them through 90 degrees as second closing means to once more close the middle section of the main diaper section.

Regions of the second closing means which serve to subsequently close the middle section of the main diaper portion, can e.g. be configured at their longitudinal end regions, approximately in the region of overlap with the hip belt. Towards this end, arbitrary gluing or mechanically closing elements can be utilized which can cooperate with the inner side or outer side of the hip belt in a gluing or bonding fashion.

It has, however, turned out to be advantageous in the event that the second closing means do not only cooperate with the hip belt rather alternatively or additionally with bordering regions of the main diaper portion in order to once more close the separation line following initial folding out or folding away of the middle diaper section from the main diaper portion. It has turned out to be advantageous in the event that the second closing means have a length in the longitudinal direction of the diaper of at least 3 to 20 cm, in particular 5 to 15 cm, in particular 7 to 15 cm.

It has furthermore turned out to be advantageous in the event that the second closing means remain on the middle section of the main diaper portion when the middle diaper section is properly folded away or flapped away and can be pushed to bond on a cooperating region in the bordering location of the main diaper portion when the middle section is subsequently closed.

The first and also the second closing means can, in and of themselves, be conventional mechanically operating closing means in particular hook and loop materials or gluing regions. Both gluing as well as mechanically acting closing elements are well known in the art so that a detailed description thereof is unnecessary. One would simply like to mention that the first closing means but also the second closing means and the associated cooperating elements on the main diaper portion or on the hip belt can be configured in such a fashion that the user can decide where to effect the bonding connection and with which tensile force it should be applied. The first and/or the second closing means can be closed at pre-propositioned cooperating closing elements or closing regions on the hip belt or on the main diaper portion. In this manner, the user can effect as good a positioning of the closing means as possible. In this event, an elastic, flexible configuration of the materials used, in particular for the closing means, has turned out to be advantageous in order to accommodate different body sizes or shapes of the user.

A further configuration of the invention proposes that the second closing means, when dispensed to the user in particular in the packaged state prior to use, are associated with the hip belt or the main diaper portion in such a fashion that they can remain in this configuration without being used and without interference. They can also be provided along with the packaged disposable diaper, separated from the hip belt and the main diaper portion. For example, in the event that the disposable diaper configured in accordance with the invention is to be used as a pure incontinence article or for certain nursing care situations which require rapid application or removal of the hygienic article, in particular by the users themselves, it can be advantageous to separately include the second closing means in the manner described.

In order to bind the absorption body of the main diaper portion to the supporting materials, it has turned out to be advantageous in the event that the intended breaking lines or separation cuts extend in the transverse direction outside of the absorption body so that, when the middle section of the main diaper section is folded or flapped away, the absorption body is folded away therewith.

It is furthermore proposed that the disposable diaper has conventional elastification means extending in the longitudinal direction in a transverse direction. The elastification means are preferentially located outside and/or inside of the intended breaking lines or separation cuts in the main diaper portion.

It has moreover turned out to be particularly advantageous in the event that the first and the second closing means have mutually overlapping strip sections. In this fashion, both the first and second closing means can be applied to the main diaper section during production in one simple application step. The first and the second closing means can be separated as longitudinal sections of a previously produced flat material web.

In accordance with an advantageous embodiment of the invention, the second closing means include a flat material strip section which bridges over the intended breaking line or separation cut. It is preferentially attached to the middle section, in particular glued. A region thereof extending in the transverse outer direction away from and past the middle section is provided with a glue layer or mechanically acting closing element disposed on the inner side of the flat material strip section facing the body. They overlap at this location with associated first closing means which are also fashioned from a flat material strip section. The first closing means form an application zone for the second closing means in the region of their overlap and on their outer side are facing away from the body. The first closing means include a glue layer or mechanically acting closing element for cooperation with the hip belt and disposed on a region which protrudes in the transverse direction past the main diaper section and preferentially on the inner side facing the body. The longitudinal extent of the first closing means preferentially assumes values of 1 to 5, in particular 1 to 4, in particular 1 to 3 cm. In contrast thereto, it has turned out to be advantageous when the second closing means, as mentioned above, preferentially extend along the entire longitudinal length of the intended breaking line or of the separation cut.

In an additional concept in accordance with the invention which is in and of itself inventive and which can be used with the disposable diaper of the above mentioned kind, the portions of the hip belt which extend in the transverse direction away from the main diaper body are configured in a pleated (Z-shaped) fashion and are folded onto themselves and/or in the direction towards a longitudinal middle axis of the disposable diaper. In a further improvement of this independent inventive concept, the sections of the hip belt which extend outwardly from both sides of the main diaper portion are folded onto each other in overlapping fashion in the direction towards the longitudinal middle axis of the diaper upon presentation of the diaper to the user, in particular in a package state prior to use. Towards this end, the free ends preferentially extend in an outward direction, i.e. away from a longitudinal axis from the diaper so that they can be easily grasped by a user of the disposable diaper to unfold the hip belt.

In a further configuration of this concept in accordance with the invention, the hip belt is made from one single material section extending in the peripheral direction. In a further configuration of this concept in accordance with the invention, the hip belt is preferentially made from a single piece and is folded towards the inside of the main diaper portion in such a fashion that one fold of one belt section is interposed in a fold of another belt section.

Further features, details and advantages of the invention can be extracted from the associated claims and the representation in the figures as well as from the subsequent description of preferred embodiments of the disposable diaper in accordance with the invention.

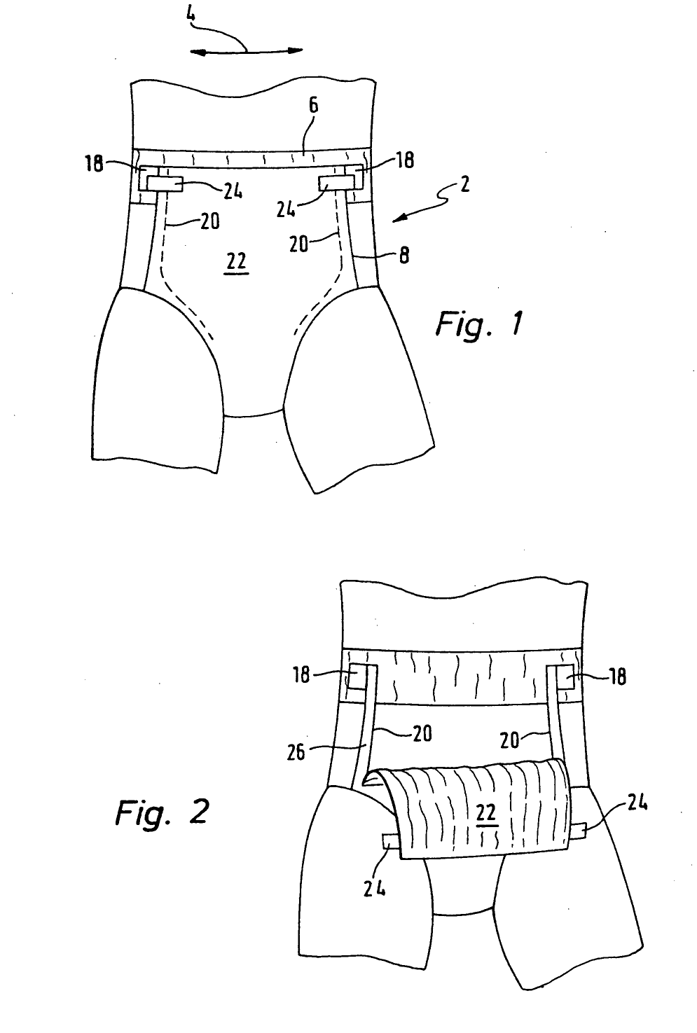

FIG. 1 shows a perspective view of a disposable diaper in accordance with the invention in the applied state;

FIG. 2 shows a perspective view of the disposable diaper in the applied state of FIG. 1, wherein a middle section of the main diaper portion is folded or clipped away from the hip belt;

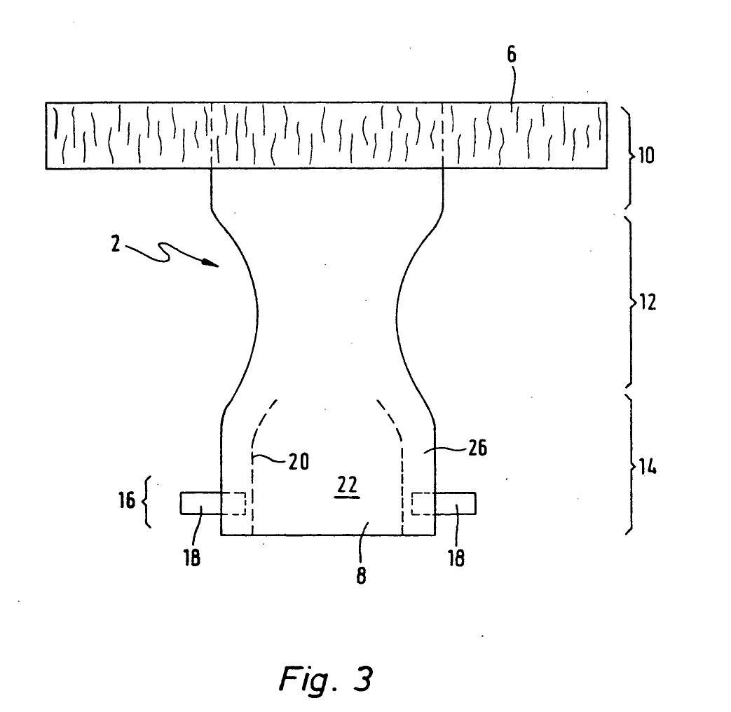

FIG. 3 shows a plan view onto the disposable diaper according to FIGS. 1 and 2 in the flat state;

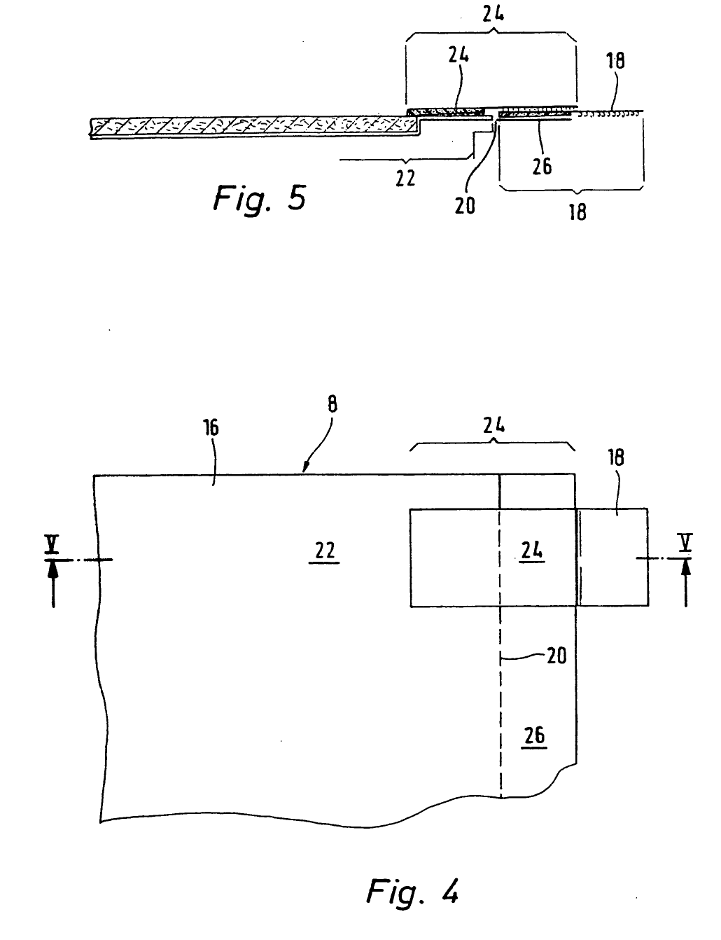

FIG. 4 shows a plan view onto a partial region of the main diaper portion in accordance with a preferred embodiment of the invention;

FIG. 5 shows a section seen in the direction of arrow V-V of FIG. 4;

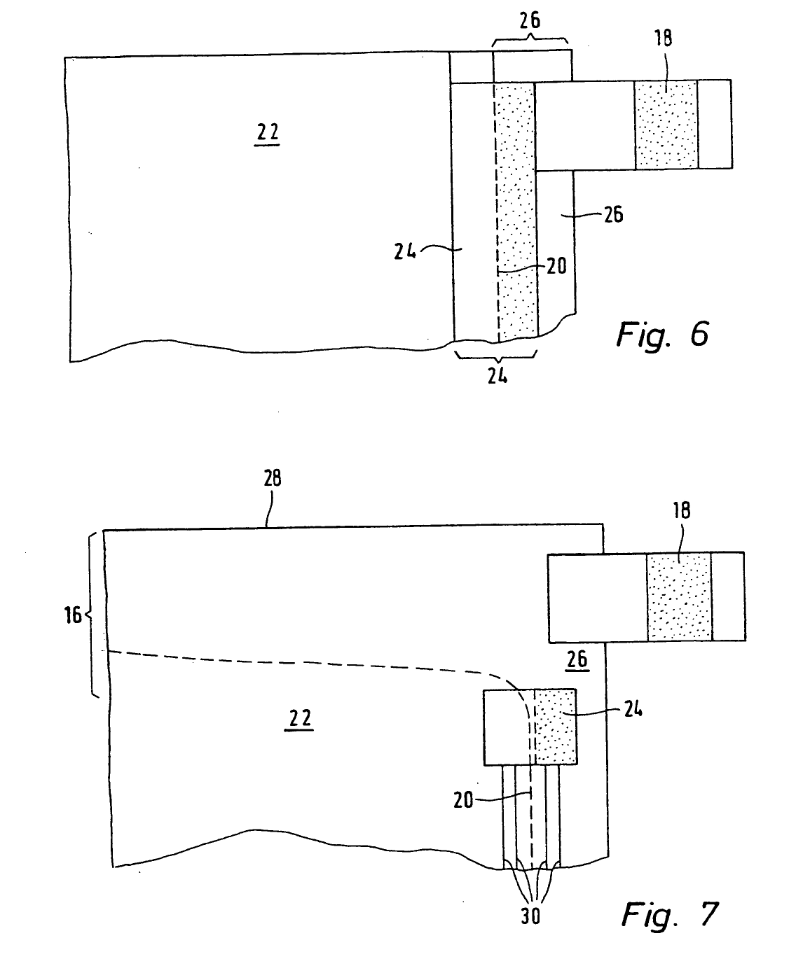

FIG. 6 shows a plan view onto a partial region of the main diaper portion in an additional embodiment of the disposable diaper in accordance with the invention;

FIG. 7 shows a plan view of a partial region of a main diaper portion of an additional embodiment of the disposable diaper in accordance with the invention.

FIGS. 1 and 2 show a disposable diaper 2 in accordance with the invention having a hip belt 6 extending in a peripheral direction 4 which can be opened and closed onto itself at at least one location. A user applies the hip belt 6 about the hips and closes it in order to attach the disposable diaper 2. The user then grasps the freely hanging main diaper portion 18 between the legs and lifts same in a forward direction for positioning in the region of the stomach on the outer side of the hip belt 6.

FIG. 3 shows a plan view of the disposable diaper in a flattened state. A back region 10 of the main diaper portion 8 is preferentially permanently attached to the hip belt 6. In addition to the rear region 10, the main diaper portion also includes a crotch region 12 and a front region 14. When applying the disposable diaper, as described above, the longitudinal end regions 16 of the front region 14 of the main diaper portion 8 overlap the outer side of the hip belt 6. In the example shown, tab-shaped first closing means 18 extend in the transverse direction 4 on both sides of the main diaper portion for attachment of the main diaper portion 8 to the hip belt 6 in a detachable fashion.

As can be seen in FIGS. 1 through 3, intended breaking lines 20 or separation cuts are provided in the front region 14 of the main diaper portion which extend from the edge of the longitudinal end region 16 in the front region 14 of the main diaper portion 18 towards the crotch region. As shown in an exemplary fashion, they initially run substantially parallel to each other and then approach each other. These intended breaking lines 20 are formed by perforations. They are initially closed when the disposable diaper is presented to the consumer. As can be seen in FIG. 2, the intended breaking lines 20 can be separated departing from a longitudinal end region 16 of the main diaper portion 8, so that a middle section 22 of the main diaper portion disposed between these intended breaking lines 20 (see FIG. 2) can be folded or flapped away from the hip belt 6. In this fashion, nursing personnel or the user can inspect the applied, disposable diaper. If, as shown, the intended breaking lines 20 or separation cuts extend to a sufficient extent in the direction towards the crotch region 12, the middle section 22 can be folded to a sufficient extent away from the hip belt 6 that a user can grasp himself in the usual manner to urinate in an outward direction. Subsequent thereto, the middle section 22 can be folded once more in an upward direction and can be attached in a detachable fashion to bordering regions 26 of the main diaper portion 8 using second closing means 24. These bordering regions (see in particular FIG. 2) are regions of the main diaper portion 8 remaining on the body of the user which are disposed on the other side of the intended breaking line 20 or of the separation cut and which are attached to the hip belt 8 in a detachable manner via the first closing means 18. FIG. 4 and the section of FIG. 5 show an embodiment with which the first and second closing means 18, 20 partially overlap each other.

In the example shown, one notices that regions 26 of the main diaper portion 8 which border the middle section 22 and the intended breaking lines 20 are shaped in a strip like or bridge like fashion, i.e. they are extended a longitudinal direction. In contrast to their longitudinal extension of approximately 10 to 50 cm, these members have a relatively narrow widths of only approximately 2 to 4 cm, in particular 2 to 3 cm, wherein these numbers are purely exemplary. The intended breaking lines 20 end at a longitudinal separation of at least 5 cm from a middle transverse line of the main diaper portion 8 which separates the overall length of the main diaper portion into two equal portions. The second closing means 24 are formed as tape sections or tabs which are permanently attached to the middle diaper section flap 22 of the main diaper section 8. They extend in the peripheral or transverse direction 4, past the side edge of the middle section 22 and therefore cover the bordering regions 26 of the main diaper portion 8. The second closing means 24 overlap the first closing means 18, which are also fashioned from a tape or a tab (see FIG. 5). The outer sides of first closing means 18 thereby constitute an impact region for the second closing means 24. First closing means 18 are attached in a permanent manner to the outer side of the region 26 bordering the middle section and are overlapped at this location by the second closing means 24. They extend in the peripheral or transverse direction 24, pass the main diaper portion 8 and can be attached in this region at an impact region of the hip belt 6 in a detachable fashion.

The detachable bonding region of the first closing means 18 and also of the second closing means 24 can be formed in an arbitrary manner either by gluing or mechanically, in particular, through conventional hook and loop materials.

The embodiment according to FIG. 6 is distinguished from the embodiments of FIGS. 4 and 5 in that the second closing means extend in the longitudinal direction along the entire length of the intended breaking lines to overlap and cover same. The second closing means are removed from the bordering region 26 of the main diaper portion in order to separate the intended breaking lines 20 and flap open the middle section 22 of the main diaper portion (see FIG. 2).

The embodiment according to FIG. 7 is particularly differentiated from the preceding embodiments in that the intended breaking lines 22 do not extend from the edge 28 on the longitudinal end of the front region 14 of the main diaper portion 8, rather run in the longitudinal end region 16 at a separation from this edge 28 in an inward peripheral direction 4. The two intended breaking lines 20 defining the middle section 22 thereby extend in an inward direction and meet each other. They therefore have the shape of an inverse “U”. In the example shown in FIG. 7, the second closing means 24 are separated from the first closing means 18 in the longitudinal direction.

In all embodiments, the elastic elements 30 extend both inside as well as outside of the intended breaking line or of a separation cut.

1-17. (canceled)

18. A disposable diaper for incontinent care, the diaper comprising:
a hip belt, said hip belt defining a closed hip opening in a peripheral direction and having means for opening and closing said hip belt at at least one location;
a main diaper portion, said main diaper portion having a front region, a rear region, an intermediate crotch region, a liquid impermeable back sheet facing away from a user's body, a liquid permeable top sheet facing the user's body, and an absorption body for bodily fluids, said main diaper portion also having two intended breaking lines or separation cuts disposed in said main diaper portion and extending from a longitudinal end region of said main diaper portion towards said crotch region, wherein a middle section of said main diaper portion disposed between and defined by said intended breaking lines or separation cuts is structured and dimensioned to fold or open away from said hip belt along said intended breaking lines or separation cuts in said main diaper portion;
first closing means for attaching said main diaper portion to said hip belt in a detachable manner at a longitudinal end of said front region or of said rear region such that, with said hip belt applied, a user raises said main diaper portion between the legs and attaches a free longitudinal end of said main diaper portion to said hip belt in a detachable fashion; and
second closing elements communicating with said middle section to attach said middle section to said hip belt or to bordering regions of said main diaper portion to once more close the diaper after said middle section has been folded or opened away.

19. The disposable diaper of claim 18, wherein said middle section of said main diaper portion has a width in a transverse direction between said intended breaking lines or separation cuts of 5 to 50 cm, in particular 5 to 40 cm, 5 to 30 cm, 5 to 25 cm, 8 to 25 cm, 10 to 25 cm, or between 15 and 25 cm.

20. The disposable diaper of claim 18, wherein a width of said middle section varies in a longitudinal direction and decreases from a longitudinal end region of said main diaper portion towards said crotch region.

21. The disposable diaper of claim 18, wherein regions of said main diaper portion bordering said intending breaking lines or separation cuts are configured in a longitudinally extended fashion.

22. The disposable diaper of claim 18, wherein regions of said main diaper portion which border said intended breaking lines or separation cuts have a width in a transverse direction of between 1 to 10 cm, between 1 to 8 cm, between 1 to 7 cm, between 1 to 6 cm, between 1 to 5 cm, between 2 to 5 cm, and between 3 and 5 cm.

23. The disposable diaper of claim 18, wherein said intended breaking lines or separation cuts have a length in a longitudinal direction of 10 to 60 cm, between 10 to 50 cm between 10 to 40 cm, between 15 to 40 cm or between 20 to 40 cm.

24. The disposable diaper of claim 18, wherein said intended breaking lines or separation cuts are separated in a longitudinal direction from a middle transverse line of said main diaper portion by at least 1 cm, at least 2 cm, at least 3 cm, at least 4 cm, or at least 5 cm.

25. The disposable diaper of claim 18, wherein said second closing means bridge over said intended breaking lines or separation cuts.

26. The disposable diaper of claim 25, wherein said second closing means bridge over said intended breaking lines or separation cuts along an entire length thereof.

27. The disposable diaper of claim 18, wherein said second closing means have a length in a longitudinal direction of the diaper of 3 to 20 cm, between 5 to 50 cm, or between 7 to 15 cm.

28. The disposable diaper of claim 18, wherein regions of said main diaper portion bordering said intended breaking lines or separation cuts form an impact region for said second closing means.

29. The disposable diaper of claim 18, wherein said intended breaking lines are formed by weakened locations or perforations.

30. The disposable diaper of claim 18, wherein said intended breaking lines or separation cuts extend, in a transverse direction of the diaper, outside of said absorption body.

31. The disposable diaper of claim 18, further comprising elastification means extending in a longitudinal direction and disposed, in a transverse direction, outside or inside said intended breaking lines or separation cuts.

32. The disposable diaper of claim 18, wherein said first and said second closing means have mutually overlapping strip sections.

33. The disposable diaper of claim 18, wherein an inner side of said first closing means facing the body seats against an outer side of said hip belt facing away from the body.

34. The disposable diaper of claim 18, wherein an inner side of said second closing means facing the body seats against an outer side of regions of said main diaper portion facing away from the body and bordering said intended breaking lines or separation cuts.
